(19) United States
(12) Patent Application Publication
(10) Pub. No.: US 2014/0187880 A1
(43) Pub. Date: Jul. 3, 2014

(54) APPARATUS AND METHOD FOR MEASURING PHYSIOLOGICAL SIGNAL

(71) Applicant: INDUSTRIAL TECHNOLOGY RESEARCH INSTITUTE, Hsinchu (TW)

(72) Inventors: Tsung-Min Hsieh, Tainan City (TW); Chen-Liang Lin, Chiayi City (TW); Jun-Chao Zhao, Tainan City (TW); Hung-Sen Tsao, Hsinchu City (TW)

(73) Assignee: INDUSTRIAL TECHNOLOGY RESEARCH INSTITUTE, Hsinchu (TW)

(21) Appl. No.: 14/050,979

(22) Filed: Oct. 10, 2013

(30) Foreign Application Priority Data
Dec. 28, 2012 (TW) 101151120

(51) Int. Cl. A61B 5/1455

(52) U.S. Cl. CPC A61B 5/14552
USPC 600/323

(57) ABSTRACT

A method for measuring a physiological signal is provided. The method is applicable to optical physiological measurement with at least two types of light sources. The method includes a processing for adjusting amplitudes of signals of the at least two types of light sources to a predetermined ratio by adjusting intensities of the light sources, so as to increase a signal dynamic range as well as a signal-to-noise ratio.

This application claims the benefit of Taiwan application Serial No. 101151120, filed Dec. 28, 2012, the disclosure of which is incorporated by reference herein in its entirety.

### TECHNICAL FIELD

The disclosure relates to an apparatus and method for measuring a physiological signal.

### BACKGROUND

A blood oxygen level represents a saturation level of oxygen contained in hemoglobin in blood, and thus indicates whether the heart and lungs are functional. In a respiratory system, carbon dioxide in the alveoli and blood is discharged in exchange with oxygen inhaled into a human body to achieve normal and balanced body functions. The ability in transporting oxygen in blood is dependent on heart functions. The blood oxygen level of a human body will be decreased by heart and thoracic cavity problems. The measurement of blood oxygen is currently performed by a pulse oxygen concentration measurement method.

Signal quality of a blood oxygen measurement apparatus greatly affects a measured value of blood oxygen concentration, and is also closely related to the amount of energy obtained from a light source transmitted through a physiological tissue. In measurement, amplitudes of energy obtained from two light sources transmitted through a physiological tissue may result in a large difference in the two optical signals due to different tissues or different measurement takers. Therefore, an automatic gain amplifier for signal amplification is usually needed. However, in the event of an extremely small signal between the two optical signals, it is likely that one single automatic gain amplifier may fail to amplify both of the optical signals to greater amplitudes, leading to a limited dynamic range of the signals.

In physiological signal measurement, taking blood oxygen concentration for example, a light source is commonly designed with a constant driving current ratio, such that small optical signals are possibly obtained due to individual differences.

### SUMMARY

The disclosure is directed to an apparatus and method for measuring a physiological signal.

According to one embodiment, an apparatus for measuring a physiological signal is provided. The apparatus comprises at least two types of light sources, at least one light source detector, at least one light source driver and a signal processing circuit.

In the apparatus according to one embodiment, the light source driver drives the at least two types of light sources according to a plurality of a signal of at least two types of initialization signals and a plurality of one other signal of the at least two types of initialization signals to render the at least one optical detector to correspondingly output a plurality of a signal of at least two types of reception signals and a plurality of one other signal of the at least two types of reception signals. The at least one light source driver drives the at least two types of light sources according to a signal of one of at least two types of operation driving signals and one other signal of the at least two types of operation driving signals.

In the apparatus according to one embodiment, the signal processing circuit selects a signal of at least two types of candidate signals for rendering one of at the least two types of light sources to enter saturation from the plurality of the signal of the at least two types of reception signals, and selects one other signal from a plurality of the other signal of the at least two types of candidate signals. A ratio of the signal of the at least two types of candidate signals to the other signal of the at least two types of candidate signals (the signal of the at least two types of candidate signals/the other signal of the at least two types of candidate signals) is approximate to a predetermined ratio. The signal processing circuit further selects the signal of the at least two types of operation driving signals corresponding to the signal of the at least two types of candidate signals from the plurality of the signal of the at least two types of initialization signals, and selects the other signal of the at least two types of operation driving signals corresponding to the other signal of the at least two types of candidate signals from the plurality of the other signal of the at least two types of initialization signals.

According to another embodiment, an apparatus for measuring a physiological signal is provided. Taking an example of two types of light source for example, the apparatus comprises a first light source, a second light source, an optical detector, a light source driver and a signal processing circuit. In an initialization period, the light source driver drives the first light source and the second light source according to first initialization signals and second initialization signals, such that the optical detector correspondingly outputs first reception signals and second receptions signals. In a measurement period, the light source driver drives the first light source and the second light source according to a first operation driving signal and a second operation driving signal. The signal processing circuit provides the first initialization signals and the second initialization signals. The signal processing circuit selects a first candidate signal for correspondingly rendering the first light source to enter saturation from the first reception signals, and selects a second candidate signal from the second reception signals. A ratio of the second candidate signal to the first candidate signal is approximate to a predetermined ratio. The signal processing circuit further selects the first operation driving signal corresponding to the first candidate signal from the first initialization signals, and selects the second operation driving signal corresponding to the second candidate signal from the second reception signals.

According to an alternative embodiment, a method for measuring a physiological signal is provided. The method comprises steps of: providing a plurality of a signal of at least two types of initialization signals and a plurality of one other signal of the at least two types of initialization signals; according to the plurality of the signal of at least types of initialization signals and the plurality of the other signal of the at least two types of initialization signals, driving at least two types of light sources such that at least one optical detector correspondingly outputs a plurality of a signal of at least two types of reception signals and a plurality of one other signal of the at least two types of reception signals; selecting a signal of at least two types of candidate signals for correspondingly rendering one of the at least two types of light sources to enter saturation from the plurality of the signal of the at least two types of reception signals, and selecting one other signal of the at least two types of candidates signals from the plurality of the other signal of the at least two types of reception signals, wherein a ratio of the signal of the at least two types of candidate signals to the other signal of the at least two types of candidate signals (the signal of the at least two types of candidate signals/the other signal of the at least two types of candidate signals) is approximate to a predetermined ratio; selecting a signal of at least two types of operation driving signals corresponding to the signal of the at least two types of candidate signals from the plurality of the signal of the at least two types of initialization signals, and selecting one other signal of the at least two types of operation driving signals corresponding to the other signal of the at least two types of candidate signals from the plurality of the other signal of the at least two types of initialization signals; and driving the at least two types of light sources according to the signal of the at least two types of operation driving signals and the other signal of the at least two types of operation driving signals.

### BRIEF DESCRIPTION OF THE DRAWINGS

FIG. 1 is a schematic diagram of a physiological signal measurement apparatus in an initialization period according to a first embodiment.

FIG. 2 is a flowchart of a physiological signal measurement method according to a first embodiment.

FIG. 3 is a timing diagram of first initialization signals and second initialization signals.

FIG. 4 is a timing diagram of first reception signals and second reception signals.

FIG. 5 is a schematic diagram of a physiological signal measurement apparatus in a measurement period according to a first embodiment.

FIG. 6 is a timing diagram of a digital signal outputted by an ADC in a delay period, an initialization phase and a measurement phase.

FIG. 7 is an enlarged partial view of T3 in FIG. 6.

FIG. 8 is a schematic diagram of a ratio of second reception signals to first reception signals.

FIG. 9 is a schematic diagram of a physiological signal measurement apparatus in an initialization period according to a second embodiment.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

Compared to infrared light, red light has a less transmission ability on a human body and thus renders lower signals. Assuming that an optimal driving ratio between energies of two light sources can be achieved with appropriate light source energies, amplitudes of the two sets of signals can be approximated to increase a signal dynamic range as well as a signal-to-noise ratio (SNR).

### First Embodiment

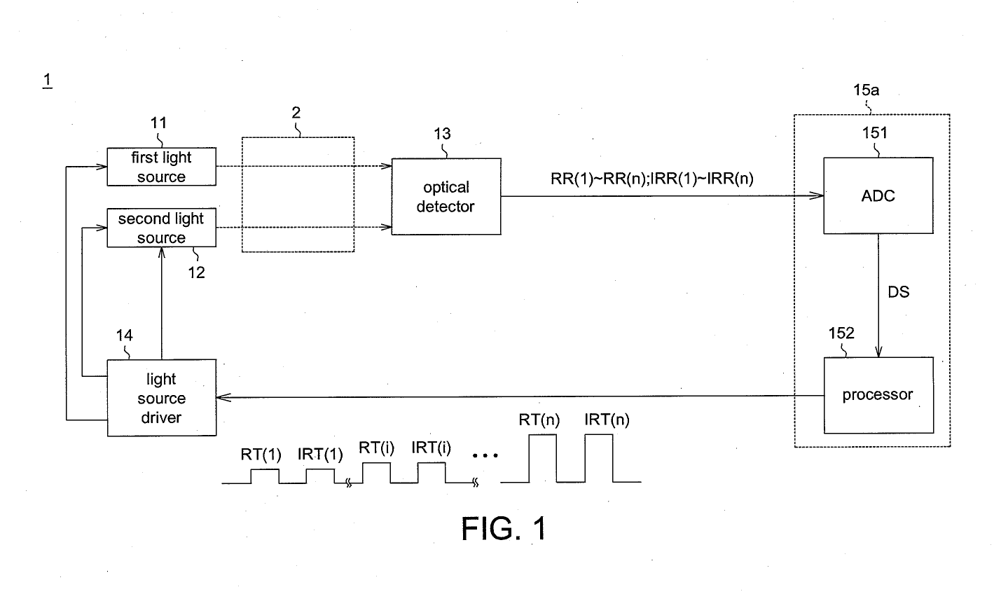

FIG. 1 shows a schematic diagram of a physiological signal measurement apparatus 1 in an initialization period according to a first embodiment. The physiological signal measurement apparatus 1, such as the blood oxygen measurement apparatus, at least comprises a first light source 11, a second light source 12, an optical detector 13, a light source driver 14, and a signal processing circuit 15a. The signal processing circuit 15a at least comprises an analog-to-digital converter (ADC) 151 and a processor 152. For example, the processor 152 is a field programmable gate array (FPGA). For example, the first light source 11 is an invisible light source and the second light source 12 is a visible light source. Alternatively, the first light source 11 may be a visible light source, and the second light source 12 may be an invisible light source. For example, the invisible light source is an infrared light light-emitting diode (LED), and the visible light source is a red light LED. For illustration purposes, in the first embodiment, the first light source 11 is a visible light source exemplified by a red light, and the second light source 12 is an invisible light source exemplified by an infrared light. The physiological signal may include blood oxygen concentration, blood sugar, carbon monoxide in blood, carbon dioxide in blood, oxidized hemoglobin (methemoglobin), hemoglobin, a heart rate, a respiratory rate, a body movement and a body temperature.

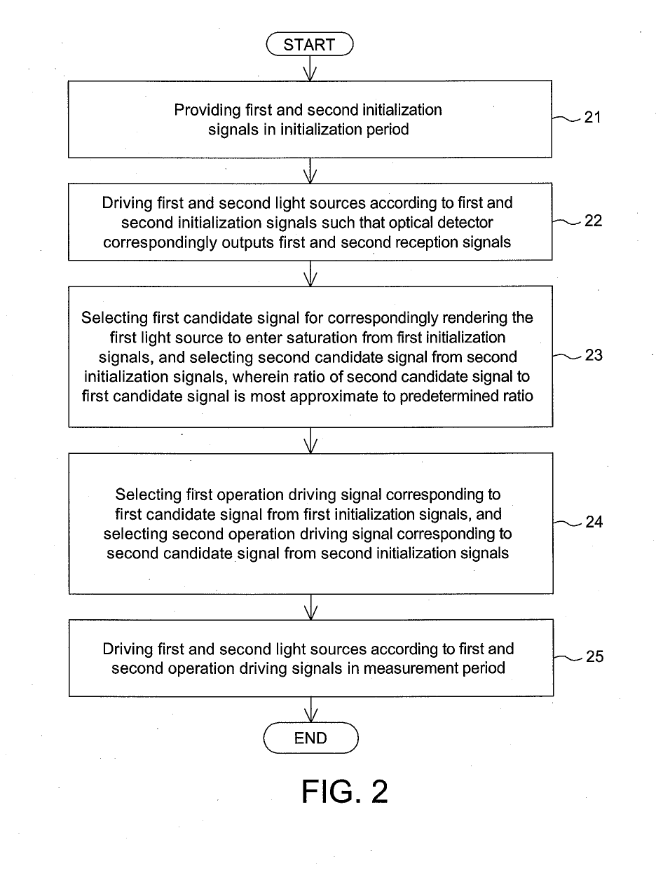

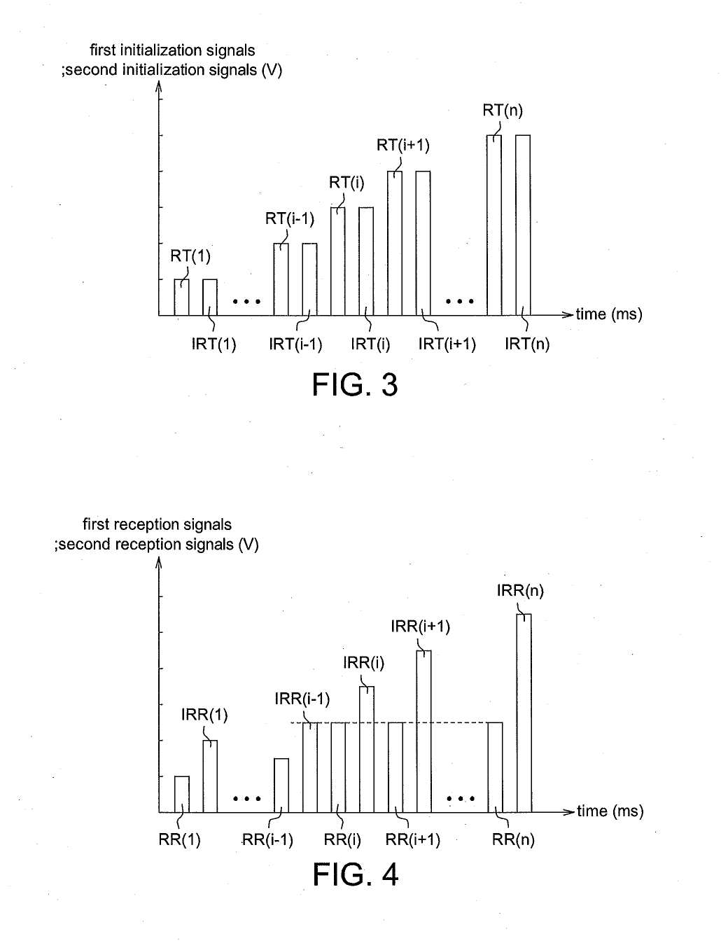

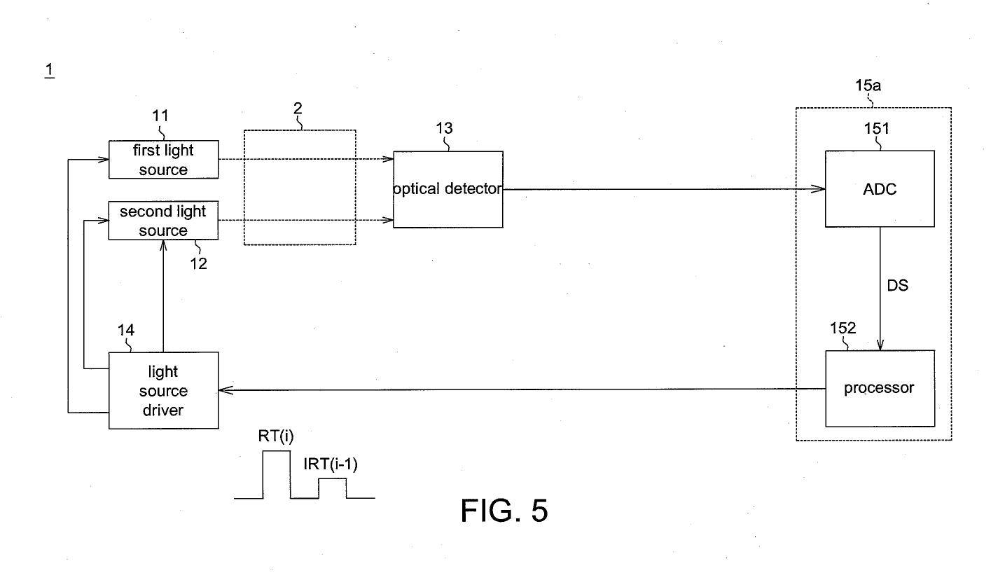

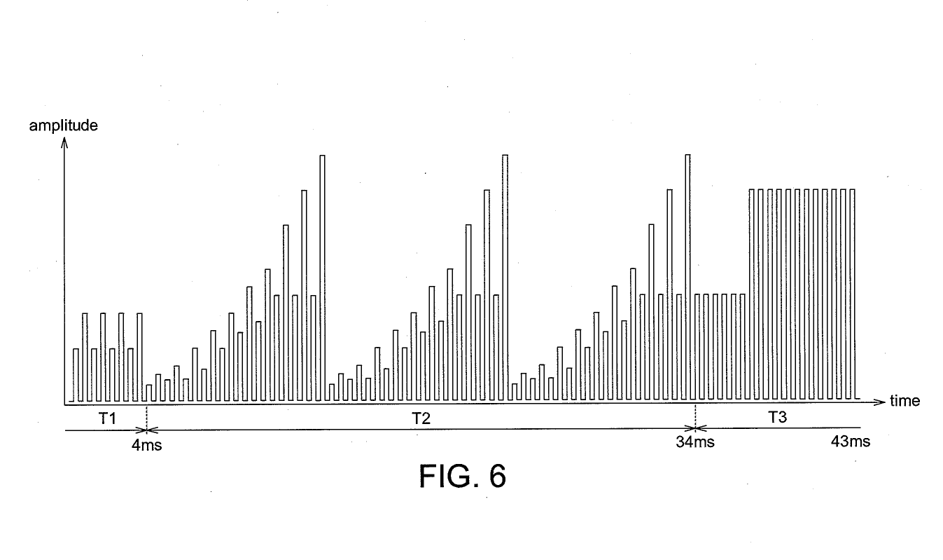

FIG. 2 shows a flowchart of a physiological signal measurement method according to the first embodiment. FIG. 3 shows a timing diagram of first initialization signals and second initialization signals according to the first embodiment. FIG. 4 shows a timing diagram of first reception signals and second reception signals. FIG. 5 shows a schematic diagram of the physiological signal measurement apparatus in a measurement period according to the first embodiment. Referring to FIGS. 2 to 5, the physiological signal measurement method, applicable to the physiological signal measurement apparatus 1, comprises the following steps.

In the initialization period, a plurality of a signal of at least two types of initialization signals and a plurality of one other signal of the at least two types of the initialization signals are provided. The signals of one of the at least two types of initialization signals may be first initialization signals RT(1) to RT(n), and the signals of the other of the at least two types of initialization signals may be second initialization signals IRT(1) to IRT(n). Referring to FIG. 2, as shown in step 21, in the initialization period, the signal processing circuit 15a provides the first initialization signals RT(1) to RT(n) and the second initialization signals IRT(1) to IRT(n). Referring to FIG. 3, for example, the first initialization signals RT(1) to RT(n) and the second initialization signals IRT(1) to IRT(n) are sequentially incremental, and the first initialization signals RT(1) to RT(n) are respectively equal to the second initialization signals IRT(1) to IRT(n). For example, the signal processing circuit 15a alternately provides the first initialization signals RT(1) to RT(n) and the second initialization signals IRT(1) to IRT(n).

According to the plurality of the signal of the at least two types of initialization signals and the plurality of the other signal of the at least two types of initialization signals, at least two types of light sources are driven such that at least one light source driver correspondingly outputs a plurality of a signal of at least two types of reception signals and a plurality of one other signal of the at least two types of receptions signals. The at least two types of light sources may be the first light source 11 and the second light source 12. The signals of one of the at least two types of reception signals may be first reception signals RR(1) to RR(n), and the signals of the other of the at least two types of reception signals may be IRR(1) to IRR(n). As shown in step 22, the light source driver 14 drives the first light source 11 and the second light source 12 according to the first initialization signals RT(1) to RT(n) and the second initialization signals IRT(1) to IRT(n), such that the optical detector 13 correspondingly outputs the first reception signals RR(1) to RR(n) and the second reception signals IRR(1) to IRR(t). It should be noted that, light beams produced by the first light source 11 and the second light source 12 transmit through a physiological tissue 2 to reach the optical detector 13. Alternatively, the light beams produced by the first light source 11 and the second light source 12 are reflected by the physiological tissue 2 to reach the optical detector 13.

A signal of at least two types of candidate signals for rendering one of the at least two types of light sources to enter saturation is selected from the plurality of the signal of the at least two types of reception signals, and one other signal of the least two types of candidate signals is selected from the plurality of the signal of the at least two types of reception signals. A ratio of the signal of the at least two types of candidate signals to the other signal of the at least two types of candidate signals (the signal of the at least two types of candidate signals/the other signal of the at least two types of candidate signals) is approximate to a predetermined ratio. The signal of the at least two types of candidate signals may be a first candidate signal, and the other signal of the at least two types of candidate signals may be a second candidate signal. As shown in step 23, from the reception signals RR(1) to RR(n), the signal processing circuit 15a selects a first reception signal RR(i) for correspondingly rendering the first light source 11 to enter saturation as a first candidate signal; from the second reception signals IRR(1) to IRR(n), the signal processing circuit 15a selects a second reception signal IRR(i−1) as a second candidate signal. A ratio of the second candidate signal IRR(i−1) to the first candidate signal RR(i) is most approximate to a predetermined ratio, e.g., 0.5 to 2. In an alternative embodiment, the predetermined ratio may be 0.8 to 1.2.

For illustration purposes, the predetermined ratio in the first embodiment is 1, for example. As the first initialization signal RT(i) already renders the first light source 11 to enter saturation, the first receptions signals RR(i+1) to RR(n) do not increase even if the light source driver 14 drives the first light source 11 according to the incremented first initialization signals RT(i+1) to RT(n). When the predetermined ratio is set to 1, the second candidate signal is most approximate to the first candidate signal. That is to say, an amplitude of the second reception signal IRR(i−1) is most approximate to an amplitude of the first reception signal RR(i).

The ADC 151 converts the first receptions signals RR(1) to RR(n) and the second reception signals IRR(1) to IRR(n) to digital signals DS, according to which the processor 152 selects the first candidate signal and the second candidate signal.

A signal of at least two types of operation driving signals corresponding to the signal of the at least two types of candidate signals is selected from the plurality of the signal of the at least two types of initialization signals, and one other signal of the at least two types of operation driving signals corresponding to the other signal of the at least two types of candidate signals is selected from the plurality of the other signal of the at least two types of initialization signals. The signal of the at least two types of operation driving signals may be a first operation driving signal, and the other signal of the at least two types of operation driving signals may be a second operation driving signal. As shown in step 24, from the first initialization signals RT(1) to RT(n), the signal processing circuit 15a selects the first initialization signal RT(i) corresponding to the first candidate signal as the first operation driving signal; from second initialization signals IRT(1) to IRT(n), the signal processing circuit 15a selects the second initialization signal IRT(i−1) corresponding to the second candidate signal as the second operation driving signal.

In a measurement period, at least two light sources are driven according to the signal of the at least two types of operation driving signals and the other signal of the at least two types of operation driving signals. As shown in step 25, in the measurement period, the signal processing circuit 15a drives the first light source and the second light source according to the first operation driving signal and the second operation driving signal. Before the measurement period, the signal processing circuit 15a has already identified the first operation driving signal and the second operation driving signal most appropriate for respectively driving the first light source 11 and the second light source 12, so that a limited dynamic range of the ADC 15 in subsequent processes is avoided.

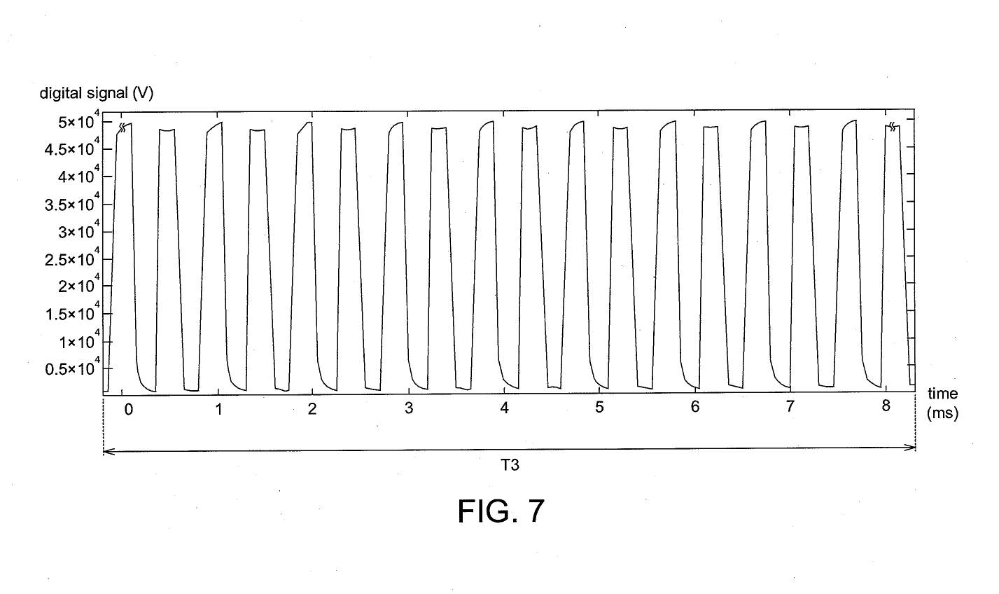

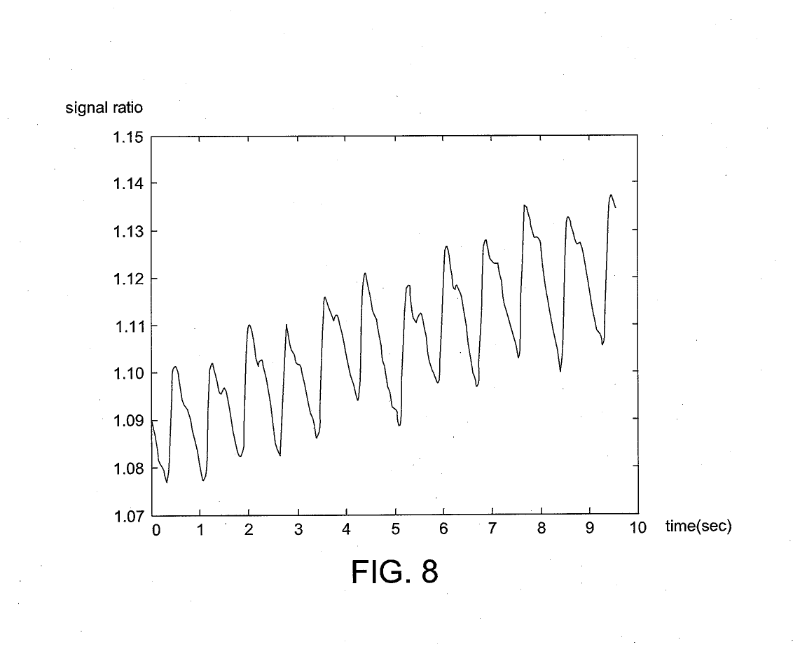

FIG. 6 shows a timing diagram of a digital signal outputted by an ADC in a delay period, an initialization phase and a measurement phase. FIG. 7 shows an enlarged partial view of T3 in FIG. 6. FIG. 8 shows a schematic diagram of a ratio of a second reception signal to a first reception signal. Referring to FIGS. 1, 2, 6, 7, and 8, the ADC 151 sequentially outputs the digital signal DS in a delay period T1, an initialization phase T2 and a measurement period T3. After powering on and the delay period T1, the physiological signal measurement apparatus 1 enters a ready state. To identify the most appropriate first operation driving signal and second operation driving signal, the physiological signal measurement apparatus 1 first performs the above steps 21 and 24 in the initialization phase T2. To further ensure the correctness of the identified first operation driving signal and second operation driving signal, steps 21 and 24 can be repeated several times. In FIG. 3, steps 21 and 24 are repeated for three times, for example.

Referring to FIG. 7, when the physiological signal measurement apparatus 1 is in the measurement phase T3, the amplitude of the digital signal DS outputted by the ADC 151 appears consistent. That is to say, when the processor 152 drives the first light source 11 and the second light source 12 according to the first operation driving signal and the second operation driving signal, the signal amplitude outputted correspondingly to the first operation driving signal and the second operation driving signal by the optical detector 13 is also consistent. When the physiological signal measurement apparatus 1 is in the measurement phase T3, the signal ratio outputted correspondingly to the first light source and the second light source by the optical detector 13 is maintained between 1.07 and 1.14, as shown in FIG. 8. Thus, the ADC 151 is prevented from a limited dynamic range.

### Second Embodiment

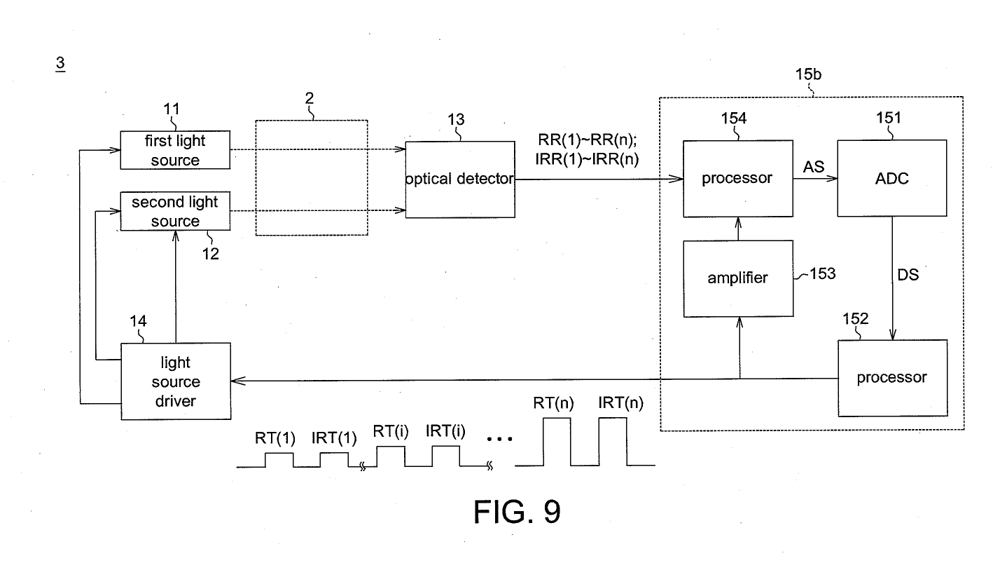

FIG. 9 shows a schematic diagram of a physiological signal measurement apparatus in an initialization period according to a second embodiment. Referring to FIGS. 1 and 9, a main difference of the second embodiment from the first embodiment is that, the signal processing circuit 15a in the first embodiment is replaced by a signal processing circuit 15b in a physiological signal measurement apparatus 3. In addition to the ADC 151 and the processor 152, the signal processing circuit 15b further comprises an auto-gain control circuit 153 and an amplifier 154. The amplifier 154 is controlled by the auto-gain control circuit 153, and amplifies the first reception signals RR(1) to RR(n) and the second reception signals IRR(1) to IRR(n) to analog signals AS. The ADC 151 converts the analog signals AS to the digital signals DS. The processor 152 selects the first candidate signal and the second candidate signal according to the digital signals DS, and selects the first operation driving signal and the second operation driving signal according to the first candidate signal and the second candidate signal. The processor 152 subsequently determines an auto-gain value of the auto-gain control circuit 153 according to the first operation driving signal and the second operation driving signal.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A method for measuring a physiological signal, comprising:
providing a plurality of a signal of at least two types of initialization signals and a plurality of one other signal of the at least two types of initialization signals;
driving at least two types of light sources according to the plurality of the signal of at least two types of initialization signals and the plurality of the other signal of the at least two types of initialization signals, such that at least one optical detector outputs a plurality of a signal of at least two types of reception signals and a plurality of one other signal of the at least two types of reception signals;
selecting a signal of at least two types of candidate signals for correspondingly rendering one of the at least two types of light sources to enter saturation from the plurality of the signal of the at least two types of reception signals, and selecting one other signal of the at least two types of candidates signals from the plurality of the other signal of the at least two types of reception signals, wherein a ratio to the signal of the at least two types of candidate signals to the other signal of the at least two types of candidate signals is approximate to a predetermined ratio;
selecting a signal of at least two types of operation driving signals corresponding to the signal of the at least two types of candidate signals from the plurality of the signal of the at least two types of initialization signals, and selecting one other signal of the at least two types of operation driving signals corresponding to the other signal of the at least two types of candidate signals from the plurality of the other signal of the at least two types of initialization signals; and
driving the at least two types of light sources according to the signal of the at least two types of operation driving signals and the other signal of the at least two types of operation driving signals.

2. The method according to claim 1, wherein the step of selecting the signal of the at least two types of candidate signals and the other signal of the at least two types of candidate signals further comprises:
converting the plurality of the signal of the at least two types of receptions signals and the plurality of the other signal of the at least two types of reception signals to a plurality of digital signals; and
selecting the signal of the at least two types of candidate signals and the other signal of the at least two types of candidate signals according to the digital signals.

3. The method according to claim 1, wherein the step of selecting the signal of the at least two types of candidate signals and the other signal of the at least two types of candidate signals further comprises:
amplifying the plurality of the signal of the at least two types of receptions signals and the plurality of the other signal of the at least two types of reception signals to a plurality of analog signals;
converting the analog signals to a plurality of digital signals; and
selecting the signal of the at least two types of candidate signals and the other signal of the at least two types of candidate signals according to the digital signals.

4. The method according to claim 1, further comprising:
determining an auto-gain value according to the signal of the at least two types of operation driving signals and the other signal of the at least two types of operation driving signals.

5. The method according to claim 1, wherein the plurality of the signal of the at least two types of initialization signals and the plurality of the other signal of the at least two types of initialization signals are sequentially incremented.

6. The method according to claim 1, wherein the predetermined ratio is between 0.5 and 2.

7. The method according to claim 1, wherein the predetermined ratio is between 0.8 and 1.2.

8. The method according to claim 1, wherein the predetermined ratio is 1.

9. The method according to claim 1, wherein providing step alternately provides the plurality of the signal of the at least two types of initialization signals and the plurality of the other signal of the at least two types of initialization signals.

10. The method according to claim 1, wherein at least one of the at least two types of light sources is an invisible light source, and one other of the at least two types of light sources is a visible light source.

11. The method according to claim 1, wherein at least one of the at least two types of light sources is a visible light source, and one other of the at least two types of light sources is an invisible light source.

12. The method according to claim 1, wherein the at least two types of light sources are visible light sources.

13. The method according to claim 1, wherein the at least two types of light sources are invisible light sources.

14. The method according to claim 1, wherein the at least two types of light sources are two light sources comprising a red light and an infrared light.

15. An apparatus for measuring a physiological signal, comprising:
at least two types of light sources;
at least one optical detector;
at least one light source driver for driving the at least two types of light sources according to a plurality of a signal of at least two types of initialization signals and a plurality of one other signal of the at least two types of initialization signals, such that the at least one optical detector outputs a plurality of a signal of at least two types of reception signals and a plurality of one other signal of the at least two types of reception signals; for driving the at least two types of light sources according to a signal of at least two types of operation driving signals and one other signal of the at least two types of driving signals; and
a signal processing circuit, for providing the plurality of the signal of the at least two types of initialization signals and the plurality of the other signal of the at least two types of initialization signals; selecting a signal of at least two types of candidate signals for correspondingly rendering one of the at least two types of light sources to enter saturation from the plurality of the signal of the at least two types of reception signals, and selecting one other signal of the at least two types of candidates signals from the plurality of the other signal of the at least two types of reception signals, wherein a ratio of the signal of the at least two types of candidate signals to the other signal of the at least two types of candidate signals is approximate to a predetermined ratio; selecting the signal of at the least two types of operation driving signals corresponding to the signal of the at least two types of candidate signals from the plurality of the signal of the at least two types of initialization signals, and selecting the other signal of the at least two types of operation driving signals corresponding to the other signal of the at least two types of candidate signals from the plurality of the other signal of the at least two types of initialization signals.

16. The apparatus according to claim 15, wherein the signal processing circuit comprises:
an analog-to-digital converter (ADC), for converting the plurality of the signal of the at least two types of receptions signals and the plurality of the other signal of the at least two types of reception signals to a plurality of digital signals; and
a processor, for selecting the signal of the at least two types of candidate signals and the other signal of the at least two types of candidate signals according to the digital signals.

17. The apparatus according to claim 15, wherein the signal processing circuit comprises:
an auto-gain control circuit;
an amplifier, controlled by the auto-gain control circuit, for amplifying the plurality of the signal of the at least two types of receptions signals and the plurality of the other signal of the at least two types of reception signals to a plurality of analog signals;
an ADC, for converting the analog signals to a plurality of digital signals; and
a processor, for selecting the signal of the at least two types of candidate signals and the other signal of the at least two types of candidate signals according to the digital signals.

18. The apparatus according to claim 17, wherein the processor determines an auto-gain value of the auto-gain control circuit according to the signal of the at least two types of operation driving signals and the other signal of the at least two types of operation driving signals.

19. The apparatus according to claim 15, wherein the signal processing circuit sequentially increments the plurality of the signal of the at least two types of initialization signals and the plurality of the other signal of the at least two types of initialization signals.

20. The apparatus according to claim 15, wherein the predetermined ratio is between 0.5 and 2.

21. The apparatus according to claim 15, wherein the predetermined ratio is between 0.8 and 1.2.

22. The apparatus according to claim 15, wherein the predetermined ratio is 1.

23. The apparatus according to claim 15, wherein the signal processing circuit alternately provides the plurality of the signal of the at least two types of initialization signals and the plurality of the other signal of the at least two types of initialization signals.

24. The apparatus according to claim 15, wherein at least one of the at least two types of light sources is an invisible light source, and one other of the at least two types of light sources is a visible light source.

25. The apparatus according to claim 15, wherein at least one of the at least two types of light sources is a visible light source, and one other of the at least two types of light sources is an invisible light source.

26. The apparatus according to claim 15, wherein the at least two types of light sources are visible light sources.

27. The apparatus according to claim 15, wherein the at least two types of light sources are invisible light sources.

28. The apparatus according to claim 15, wherein the at least two types of light sources are two light sources comprising a red light and an infrared light.
